(19) United States
(12) Patent Application Publication
(10) Pub. No.: US 2014/0023696 A1
(43) Pub. Date: Jan. 23, 2014

(54) TREATMENT FOR IDIOPATHIC PULMONARY FIBROSIS

(71) Applicant: Frederick Timothy Guilford, Palo Alto, CA (US)

(72) Inventor: Frederick Timothy Guilford, Palo Alto, CA (US)

(21) Appl. No.: 13/840,721

(22) Filed: Mar. 15, 2013

Related U.S. Application Data
(60) Provisional application No. 61/674,093, filed on Jul. 20, 2012.

(51) Int. Cl. A61K 38/06; A61K 9/127

(52) U.S. Cl. CPC A61K 38/063; A61K 9/127
USPC 424/450; 514/21.9

(57) ABSTRACT

The invention proposes a method of treatment of idiopathic pulmonary fibrosis by a liposomally formulated reduced glutathione.

### CONTINUATION DATA

This is a non-provisional application and claims benefit of U.S. Provisional application 61/674,093 filed Jul. 20, 2012.

### FIELD OF INVENTION

The invention relates to the treatment of idiopathic pulmonary fibrosis (IPF) by a novel method using liposomal reduced glutathione.

### SUMMARY

The invention proposes a method of treatment of idiopathic pulmonary fibrosis by a liposomally formulated reduced glutathione.

### BACKGROUND

“Idiopathic pulmonary fibrosis (IPF) (or cryptogenic fibrosing alveolitis (CFA) or idiopathic fibrosing interstitial pneumonia) is a chronic, progressive form of lung disease characterized by fibrosis of the supporting framework (interstitium) of the lungs. By definition, the term is used only when the cause of the pulmonary fibrosis is unknown (“idiopathic”).

Wikipedia, accessed Jul. 9, 2012 under Idiopathic_pulmonary_fibrosis.

According to the “Treatment” segment of the U.S. National Library of Medicine's PubMed Health on-line database, updated and reviewed by a team of clinicians as of May 29, 2012, accessed on Jul. 6, 2012, there is no treatment available for idiopathic pulmonary fibrosis.
“No known cure exists for idiopathic pulmonary fibrosis. Unfortunately, no medication has been shown to improve the outcome of patients with this condition.
   For some people, medications such as corticosteroids and cytotoxic drugs may help reduce swelling (inflammation). However, these drugs might also increase the risk of death.
   Other new treatments that have been shown to help some people with idiopathic pulmonary fibrosis are being studied.
   Patients with low blood oxygen levels may need oxygen.
   Lung rehabilitation will not cure the disease, but it can help maintain exercise capacity (the ability to exercise without breathing difficulty).
You can make home and lifestyle changes to manage breathing symptoms. Anyone who smokes should stop right away.
Some patients with advanced pulmonary fibrosis may need a lung transplant.”

In the segment in the same source entitled Expectations (Prognosis), the following is noted:
“Some patients may improve or stay stable for a long time when they are treated with corticosteroids or cytotoxic drugs. However, in most people the disease can get worse even with treatment. This worsening can happen quickly, or very slowly.
When breathing symptoms become more severe, discuss treatments that prolong life, health care agents, and advanced care directives with your health care provider.”

The implications of the advanced care directives are that if the disease progresses and there is no response to treatment, the disease is debilitating and fatal.

According to Wikipedia, under the subject of Idiopathic_pulmonary-fibrosis, accessed on Jun. 9, 2012,
“There is no consensus on treatment and no satisfactory treatment exists.
There is a lack of large, randomized placebo-controlled trials of therapy for IPF. Moreover, many of the earlier studies were based on the hypothesis that IPF is an inflammatory disorder, and hence studied anti-inflammatory agents such as corticosteroids. Another problem has been that studies conducted prior to the more recent classification of idiopathic interstitial pneumonias failed to distinguish IPF/UIP from NSIP in particular. Hence, many patients with arguably more steroid-responsive diseases were included in earlier studies, confounding the interpretation of their results.
A large randomized, controlled trial (PANTHER-IPF) found that the combination of prednisone, azathioprine, and N-acetylcysteine had a significantly higher death rate than placebo (8 vs. 1), and the trial was terminated.
Other treatments studied have included interferon gamma-1b, the antifibrotic agent pirfenidone and bosentan. Pirfenidone and bosentan are currently being studied in patients with IPF while interferon gamma-1b is no longer considered a viable treatment option. Finally, the addition of the antioxidant N-acetylcysteine to prednisone and azathioprine produced a slight benefit in terms of FVC and DLCO over 12 months of follow up. However, the major benefit appeared to be prevention of the myelotoxicity associated with azathioprine. [citations omitted].”

The inventor believes that the proposed method enables improved macrophage function to address and allay Idiopathic pulmonary fibrosis. Macrophages (MP's) play a significant role in the management of infected or damaged tissues. The inventor proposes that improved macrophage function would improve the immune system capacity to combat idiopathic pulmonary fibrosis. Investigation of macrophages in tumors shows them to be divided into two general groups based on the expression of cytokines by the MP's and described as M1 or M2. The classification as to M1 or M2 is determined by the expression of Interleukins, a group of cytokines (secreted proteins/signaling molecules) that are released by leukocytes (white blood cells) and act on leukocytes. A phenotype (from Greek phainein, ‘to show’+typos, ‘type’) is the composite of an organism's observable characteristics or traits. Phenotypes result from the expression of an organism's genes as well as the influence of environmental factors and the interactions between the two.

Classical macrophages, noted as M1, have been characterized as a phenotype characterized by interleukin 12-High (IL-12^{high}) Interleukin 23-High (IL-23^{high}), and interleukin 10-low (IL-10^{low}). M1 macrophages are immune effector cells that are aggressive against microbes and can engulf and digest affected cells much more readily, M1 macrophages produce reactive oxygen and nitrogen intermediates as well as inflammatory cytokines and play a role in upregulating T helper cell 1 (Th1) responses are mediated by the white blood cells that help other immune cells by activating and directing their function. They help maximize the bactericidal activity of phagocytes such as macrophages. TH1 activity functions in a manner that continues an efficient and effect macrophage cell function in terms of killing invaders such as infection, parasites and cancer cells (1).

The M2 macrophage phenotype is characterized by an IL-12^{low}, IL-23^{low}, and IL-10^{high} presentation IL-10 is involved in turning off immune system activation and helps decrease inflammation. The function of M2 macrophages is diverse, but in general they are involved in T helper 2 (Th2) response, whose main partners are B-cells which is generally associated with the production of antibodies from B-cells. M2 type macrophages have an immunoregulatory function, and orchestrate encapsulation and containment of parasites and promote tissue repair, remodeling, and tumor progression (1).

| Macrophage Type | L-12 | IL-23 | IL-10 |
|---|---|---|---|
| M1 | High | High | Low |
| M2 | Low | Low | High |

An immunological marker distinguishing macrophages from other immune cells is the marker CD68. In the immune system the type of white blood cell called lymphocytes have been found to perform different functions in immune defense. Before the function of these cells was understood, a way to identify the cells was found using antibodies specific to various clusters of proteins found on the surface of the lymphocyte. These antibodies were able to chart the different types of lymphocyte populations based on the appearance of specific immunologically distinctive protein clusters as markers. These protein markers ultimately were associated with functionally distinct populations of lymphocytes such as B-cells, helper T-cells (TH), cytotoxic T-cells (TC), and natural killer (NK) cells. These different populations have become designated by the cluster of differentiation (CD) antigen number. The first group identified was CD group 1, designated CD1. The second was designated CD2 and so on. At the time this designation was being formed, the actual function of the lymphocytes was not known. It has been subsequently shown that the white blood cells, called T helper (TH) lymphocytes always show a cluster designation number 4 and are now known as CD4. The cluster of differentiation (CD) CD68 is associated with macrophages and the presence of this marker makes it useful in diagnosing the accumulation of macrophages in various tissues.

Macrophages, from the Greek, meaning “large eaters,” are large phagocytic leukocytes, which are able to move outside of the vascular system by moving across the cell membrane of capillary vessels and entering the areas between cells in pursuit of invading pathogens. In tissues, organ-specific macrophages are differentiated from phagocytic cells present in the blood called monocytes. Macrophages are the most efficient phagocytes, and can phagocytose substantial numbers of bacteria or other cells or microbes. The binding of bacterial molecules to receptors on the surface of a macrophage triggers it to engulf and destroy the bacteria through the generation of a “respiratory burst”, causing the release of reactive oxygen species. Pathogens also stimulate the macrophage to produce chemokines, which summons other cells to the site of infection.

In cancer, macrophage infiltration around a tumor may help delay tumor development. This suggests that peritumoral tumor-associated macrophages (TAM) are associated with increased survival of the host and a better prognosis in tumors such as colon cancer. This suggests that peritumoral macrophages are of the M1 phenotype. In contrast, intratumoral TAM count has been correlated with depth of invasion, lymph node metastasis, and staging of colon and rectal cancers, suggesting that intratumoral M2 macrophages cause cancer cells to have a more aggressive behavior (1).

It has been suggested that these contradictory functions of MP's may have different additional markers. While CD68 is a general marker of MP's the use of subset markers such as CD163 or CD204 might have an increased significance. The use of CD204 as a marker of macrophages in lung adenocarcinoma has a strong association with poor outcome (2). In a similar fashion CD164+TAM has been shown to correlate with myometrial invasion in endometrial carcinoma of the uterus (3). In pancreatic cancer, high numbers of CD163- or CD204-positive macrophages were associated with poor prognosis (P=0.0171); however, this was not the case for the number of CD68-positive macrophages (4).

CD 163, a haptoglobin-hemoglobin complex is implicated as a hemoglobin scavenger receptor for binding of erythrocytes to macrophages for the removal of iron containing proteins and is expressed in monocytes and macrophages. CD163 can also function as a macrophage receptor for both Gram-positive and -negative bacteria (5) (5). Recent work has shown that this marker is also specific for neoplasms of histiocytic differentiation in the skin (6).

The inventor has hypothesized that the receptor for CD163 may preferentially attach to the liposome of the liposomal reduced glutathione in a manner similar to the absorption demonstrated by the macrophages from individuals with HIV that are undergoing infection (Unpublished data Venketaraman and Guilford, Western University 2012). The presence of CD163 appears to increase the absorption of the liposome and its glutathione content. The result of this surprising absorption of glutathione using liposomal reduced glutathione correlates clinically to the surprising and unexpected finding of resolution of the Merkel Cell Carcinoma reported in the Case Example. The invention also proposes using PEG headed thermodynamically stable liposomes made in a thermodynamically stable environment, but the lecithin based formulation may be more effective that the PEG headed formulation for absorption by macrophages.

The proposed invention, the liposomal encapsulation of reduced glutathione (also referred to as liposomal reduced glutathione (LRG), also has advantages in oxidized environments as macrophages display another cell surface marker known as CD36 in oxidized environments. CD36 functions as a scavenger receptor for lipoproteins. In an oxidizing environment low density lipoprotein can become oxidized, forming a toxic compound, oxidized low density lipoprotein LDL (oxLDL), which is taken up by the CD36 receptor. It appears that the CD36 receptor also takes up the LRG the invention to provide reduced glutathione to the interior of the cell. The delivery of reduced glutathione protects the cells, especially macrophages from damage from the toxic effects of the LDL complex, and allows the metabolic machinery of the cell to transform the oxidized lipoprotein to a more manageable form that can be handed off to high density lipoprotein (HDL) for return to the liver.

It has also been shown that IPF is related to an increase in the production of the cytokine transforming growth factor beta (TGF-β). TGF-β plays a key role in the tissue remodeling or fibrotic process observed in bronchial asthma, chronic pulmonary disease (COPD), and idiopathic pulmonary fibrosis (IPF). TGF-beta has been reported to decrease the production of intracellular glutathione and stimulate the production of reactive oxygen species. TGF-β has been shown to induce scarring and fibrosis in tissues and its tissue activity can be reversed by maintaining glutathione levels. TGF-β also induces induce epithelial-mesenchymal (fibroblast) transition (EMT) in alveolar epithelial cells (AEC). The Fibroblast transition cells can then contribute to the fibrosis found in IPF. The inventors believe supporting glutathione levels in tissues such as lung with increased levels of TGF-β will stop the action of TGF-β and slow or stop the formation of fibrosis in the lung of individuals with IPF.

The macrophages from individuals with human immunodeficiency virus (HIV) have been shown to be low in glutathione and particularly vulnerable to infection with Mycobacterium tuberculosis (the infectious agent of the disease known as Tuberculosis). An additional unpublished study shows that liposomal reduced glutathione formulated per this invention has a significantly increased absorption and function in the macrophages from individuals with HIV that are undergoing infection with M. tb. The absorption of the liposomal glutathione is 1000×'s more efficient than the glutathione precursor N-acetyl cysteine (NAC) in restoring normal glutathione levels and restoring the glutathione related function of slowing the replication of M tb in macrophages taken from individuals with HIV . . . “Glutathione Supplementation Improves Immune Function in HIV+ Macrophages,” Morris D, Guerra C, Khurasany M, Guilford T, Venketaraman V, (unpublished, Western University of Health Sciences, Pomona, Calif. 91766, USA) (“Morris D”).

The surprising and novel finding in the unpublished Morris D et al study of the dramatic absorption of liposomal reduced glutathione compared to N-acetyl cysteine (“NAC”) explains the ability of this formulated form of liposomal reduced glutathione to restore macrophage function back to the M1 function.
“In a previous study we observed elevated levels of TGF-β in both the plasma and macrophage culture supernatants of HIV+ macrophages [42]. This elevated TGF-β will compromise the amount of GCLC present inside the cell; consequently, supplementing the raw materials [such as with NAC] for de novo synthesis in HIV+ individuals who are over expressing TGF-β will not result in the same increased production of reduced GSH that is observed in individuals who are not over expressing TGF-β. In addition, this phenomenon may explain why lGSH [the liposomal reduced glutathione of this invention] at lower concentrations than NAC is more effective at raising the concentration of reduced GSH in HIV+ macrophages than in HIV− macrophages. Supplementing with an lGSH formulation provides complete GSH molecules to cells, circumventing the enzymatic pathway responsible for GSH production, without the requirement for the cell to construct the tripeptide. This may also explain why treatment with lGSH seems to raise the ratio of reduced GSH to GSSG at much lower concentrations than NAC, as cells treated with NAC will have to produce new molecules of reduced GSH utilizing their own enzymatic machinery. [emphasis added, citation omitted].” Morris et al at pp. 17-18.

### PREFERRED MODE OF INVENTION

### Preferred Dosing

For systemic adjunctive management of idiopathic pulmonary fibrosis and support of immune function in individuals with idiopathic pulmonary fibrosis.

Oral liposomal glutathione 1.5 (approximately 600 mg) teaspoons twice a day. More consistent dosing and effect occurs on an empty stomach but that is not essential to method of the invention.

Another preferred mode is to administer up to the tolerated dose as many as 4 teaspoons throughout the day to maintain consistent and adequate glutathione resources. Smaller doses more often are appropriate if a patient is initially less tolerant of the dose. Patients call build up to a maintenance dose. Oral administration by day and parenteral administration by night is also contemplated. A final amount of deionized water can be added as necessary to have percentages add up to 100% w/w.

### Formulation

Glutathione can be obtained from various sources including Kyowa Hakko U.S.A., 85 Enterprise, Suite 430, Aliso Vieja, Calif. 92656.

### Example 1

### Liposomal Glutathione Drink or Spray 2500 Mg Per Ounce or Form Suitable for Encapsulation or Gel

| | % w/w |
|---|---|
| Deionized Water | 74.4 |
| Glycerin | 15.00 |
| Lecithin | 1.50 |
| Potassium Sorbate | 0.10 |
| (optional spoilage | |
| retardant) | |
| Glutathione | 8.25 |
| (reduced) | |

A lipid mixture having components lecithin, ethyl alcohol and glycerin were commingled in a large volume flask and set aside for compounding. Hydroxylated lecithin is the preferred ingredient in each of the embodiments of the invention including this embodiment. In a separate beaker, a water mixture having water, glycerin, glutathione were mixed and heated to 50.degree.C.

The water mixture was added to the lipid mixture while vigorously mixing with a high speed, high shear homogenizing mixer at 750-1500 rpm for 30 minutes.

The homogenizer was stopped and the solution was placed on a magnetic stifling plate, covered with parafilm and mixed with a magnetic stir bar until cooled to room temperature. Normally, citrus seed extract or flavorant would be added for taste enhancement. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of glutathione. The methods of manufacture described in Keller et al, U.S. Pat. No. 5,891,465, U.S. Pat. No. 6,958,160 and U.S. Pat. No. 7,150,883 are incorporated in this description.

Concentrations of liposomal glutathione from 3.3%, 4%, 5%, 6%, 7%, 7.5%, 8%, 8.5% or 9% w/w or greater in 0.5% increments of lipoceutical glutathione may be formed and utilized for dosing by decreasing the amounts of glutathione and preplacing the material with an increase in the sterile water concentration. The amount of 3.3% w/w corresponds to a concentration of 123 mM.

### Example 2

### Liposomal Glutathione Drink or Spray 2500 Mg Per Ounce or Form Suitable for Encapsulation or Gel

| | % w/w |
|---|---|
| Deionized Water | 74.4 |
| Glycerin | 15.00 |
| Lecithin | 1.50 |
| Potassium Sorbate | 0.10 |
| (optional spoilage | |
| retardant) | |
| Glutathione | 8.50 |
| (reduced) | |

A lipid mixture having components lecithin, ethyl alcohol and glycerin were commingled in a large volume flask and set aside for compounding.

In a separate beaker, a water mixture having water, glycerin, glutathione were mixed and heated to 50.degree.C.

The water mixture was added to the lipid mixture while vigorously mixing with a high speed, high shear homogenizing mixer at 750-1500 rpm for 30 minutes.

The homogenizer was stopped and the solution was placed on a magnetic stifling plate, covered with parafilm and mixed with a magnetic stir bar until cooled to room temperature. Normally, citrus seed extract would be added. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of glutathione. The methods of manufacture described in Keller et al, U.S. Pat. No. 5,891,465, U.S. Pat. No. 6,958,160 and U.S. Pat. No. 7,150,883 are incorporated in this description.

Concentrations of liposomal glutathione from 3.3%, 4%, 5%, 6%, 7%, 7.5%, 8%, 8.5% or 9% reduced glutathione may be formed and utilized for dosing by decreasing the amounts of glutathione and replacing the material with an increase in the sterile water concentration.

Further example 3
Formulation for Topical application of liposomal reduced glutathione
Suitable pharmaceutical carriers and adjuvants (among others)

### Caprylic/Capric Triglyceride

Sweet Almond (Pruns amygdalus dulcis) oil

### PEG-12 Glyceryl Dimyristate

### PEG-23 Glyceryl Disterate

### Canola Oil

Liposomal reduced glutathione in percentages reference in this application, e.g. Glutathione (5%)

### Glyceryal Stearate

### PEG-100 Stearate

### Cetearyl Alcohol

### Polysorbate 60

### Silica

### Sea Mays (Corn) Starch

### Tocopheryl Acetate

Formulation for Topical application of liposomal reduced glutathione

A topical cream or lotion containing reduced glutathione in a self-forming liposome sold under the brand name “QuSome”® by Biozone Laboratories, Inc. of Pittsburgh, Calif. is another preferred embodiment. The Qusome self-forming liposome can be formed containing reduced liposomal glutathione in a concentration, for example, of 5% reduced glutathione in the liposome or in percentages discussed in this application. Most liposomes use energy provided as heat, sonication, extrusion, or homogenization for their formation, which gives them a high energy state. Some liposome formulations can experience problems with aggregation, fusion, sedimentation and leakage of liposome associated material which this invention seeks to minimize and does minimize. The Qusome is a more thermodynamically stable liposome formulation. The Qusome self-forming liposome is self-forming at room temperature which that the mixing of the lipid and an aqueous lipid containing solution avoids alteration of the contents by heating. The resulting liposome is in a low free energy state so it remains stable and reproducible. The formulation of this embodiment is reviewed in example 3. The methods of manufacture described in Keller et al U.S. Pat. No. 6,958,160 and U.S. Pat. No. 7,150,883 are incorporated in this description. The most important details of that manufacturing are as follows:

The lipids used to form the lipid vesicles and liposomes in the present formulations can be naturally occurring lipids, synthetically made lipids or lipids that are semisynthetic. Any of the art known lipid or lipid like substances can be used to generate the compositions of the present invention. These include, but are not limited to, lecithin, ceramides, phosphatidylethanolamine, phosphotidylcholine, phosphatidylserine, cardiolipin and the like. Such lipid components for the preparation of lipid vesicles are well known in the art, for example see U.S. Pat. No. 4,485,954, and “Liposome Technology”, 2nd Ed, Vol. I (1993) G. Gregoriadis ed., CRC Press, Boca Raton, Fla.

Lipids with these properties that are particularly preferred in the present formulations include phospholipids, particularly highly purified, unhydrogenated lecithin containing high concentrations of phosphotidylcholine, such as that available under the trade name Phospholipon 90 from American Lecithin, or Nattermann Phospholipid, 33 Turner Road, Danbury, Conn. 06813-1908.

In formulating the liposomes, In one aspect, the invention includes a method of preparing liposomes. The method comprises providing an aqueous solution; providing a lipid solution, where the solution has a Pₐ between about 0.84 and 0.88, a Pᵥ between about 0.88 and 0.93, and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolyglycerol-PEG-12, either alone or as one of the lipids in a mixture. The method may further comprise providing an active compound; and combining the active compound with the lipid solution and the aqueous solution.

In another aspect, the invention includes a liposome suspension. The suspension comprises one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain. The PEG chain preferably has a molecular weight between about 300 Daltons and 5000 Daltons. The suspension may comprise a single lipid. The lipid may comprise dioleolylglycerol-PEG-12. The suspension may further comprise an active compound, which may be selected from the group described above.

In another aspect, the invention includes a composition for combining with an aqueous solution to form a liposome suspension. The composition comprises one or more lipids, where the lipids as an aggregate have a Pₐ between about 0.84 and 0.88, a Pᵥ, between about 0.88 and 0.93 and a melting temperature of between about 0 to 100 degrees centigrade; and where at least one lipid includes a polyethyleneglycol (PEG) chain. The PEG chain preferably has a molecular weight between about 300 Daltons and 5000 Daltons. The composition may comprise a single lipid. The composition may comprise dioleolylglycerol-PEG 12. The composition may further comprise an active compound selected from the group above. The composition may be provided in a sealed container, where the container also contains an inert gas to prevent oxidative degradation.

In another aspect, the invention includes a method of intravenously administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension intravenously. The method may further comprise providing kinetic energy to the liposome suspension. The method may also include providing the composition in a sealed container containing an inert gas. The PEG chain preferably has a molecular weight between about 300 Daltons and 5000 Daltons. The composition may comprise a single lipid. The lipid may comprise dioleolylglycerol-PEG-12. The active compound may be selected from the group above.

In another aspect, the invention includes a method of solubilizing an active compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing the active compound; providing an aqueous solution; and combining the active compound, the lipid and the aqueous solution to form a liposome suspension. The method may further comprise providing kinetic energy to the liposome suspension. The method may include providing the composition in a sealed container containing an inert gas. The PEG chain preferably has a molecular weight between about 300 Daltons and 5000 Daltons. The composition may comprise, a single lipid. The lipid may comprise dioleolylglycerol-PEG-12. The active compound may be selected from the group above.

In another aspect, the invention includes a method of orally administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension orally in the form selected from the group comprising a two piece hard gelatin capsule, a soft gelatin capsule, or drops. The compositions may be administered topically, inter-orally, vaginally or rectally.

PEG-12 Glyceryl Dioleate was obtained from Global 7 (New Jersey) for the following formulations. This can be substituted for the lecithin w/w % as needed to accomplish the formulation, or applied as set forth below.

In the following formulations, the “set percentage” w/w % of reduced glutathione is selected from 3.3%, 4%, 5%, 6%, 7%, 7.5%, 8%, 8.5% or 9% or amounts approximately to those percentages.

### Example 3A

### Spontaneous Liposomes for Intravenously Administering Therapeutic Compounds or for a Spray or Drink

A set percentage of reduced glutathione is dissolved in a sufficient amount of the solvent PEG-12 Glyceryl Dioleate, also called dioleolylglycerol-PEG 12, (either referred to as “PEGDO”) and gently mixed for about 5 minutes. A sufficient amount of PEGDO should be about 10% w/w. Deionized water is slowly added to the solution. Ingredients other than deionized water, the reduced glutathione and the PEGDO may be added such as preferably 0.1% w/w potassium sorbate and then the final amount of deionized water added is that amount which is necessary to have the percentages add up to 100% w/w. Taste or other flavor-masking ingredients could also be added before the deionized water is brought up to 100% w/w. Although taste ingredients can be added before or after the liposomal formulation, the preferable mode is to add flavor or other taste masking ingredients after liposomal formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10%. The inventor has preferably used citrus seed extract.

### Example 3B

### Spontaneous Liposomes for Intravenously Administered Therapeutic Compound and as a Drug Solubilization Vehicle for Use in Spray or Drink

A set percentage of reduced glutathione is mixed with a sufficient amount of PEG-12 Glyceryl Dioleate, also called dioleolylglycerol-PEG 12, (either referred to as “PEGDO”) to bring the reduced glutathione into solution by vortexing and sonication for 10 minutes. A sufficient amount of PEGDO should be about 5% w/w but can be other percentages discussed in this application. Deionized water is added and gently mixed. Ingredients other than deionized water, the reduced glutathione and the PEGDO may be added such as preferably 0.1% w/w potassium sorbate and then the final amount of deionized water added is that amount which is necessary to have the percentages add up to 100% w/w. Ingredients other than deionized water, the reduced glutathione and the PEGDO may be added such as preferably 0.1% w/w potassium sorbate and then the final amount of deionized water added is that amount which is necessary to have the percentages add up to 100% w/w. Taste ingredients or other flavor masking ingredients could also be added before the deionized water is brought up to 100% w/w. Although taste ingredients can be added before or after the liposomal formulation, the preferable mode is to add flavor or other taste masking ingredients after liposomal formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10%. The inventor has preferably used citrus seed extract.

The QuSome self-forming liposome uses polyethyleneglycol (PEG) is a steric stabilizer and the resulting liposome is of a moderate size, 150 nm-250 nm. The combination of 150 nm-250 nm size and the PEG component is known to create long circulating liposomes. The size of the QuSome self-forming liposome allows them to be sterile filtered. These attributes allow a secondary advantage of the invention by the QuSome liposome encapsulating a radionuclide useful for targeting tumors with either diagnostic radionuclides or therapeutic radionuclides. The QuSome self-forming liposome is of such as size and the presence of the steric stability with PEG results in long circulation time and an increased accumulation in the fine trabecular mesh of blood vessels supplying growing tumors. This characteristic allows for improved diagnostics as more radionuclide accumulates around the tumor improving the image of scans. This characteristic of accumulating in the trabecular mesh of blood vessels leading to tumors also leads to an improved therapeutic. The accumulation of QuSome self-forming liposomes in the blood vessel supply to tumors increases the radiation dosing to this area, creating damage to the tumor blood vessels creating an anti-angiogenic effect, resulting in a decreased supply of blood to the tumor and leading to death of tumor cells.

The concentration of liposomal glutathione in the Qusome formulation is 5% for topical application. It is possible to use the Qusome technology in creating an oral formulation also and the 8.25% glutathione in w/w concentration may be used in the oral formulation.

Liposomal glutathione can be infused intravenously (2000 mg intravenously) and another 2500 mg (approximately 6 teaspoons of liposomal reduced glutathione) can be ingested orally. The dose of oral liposomal reduced glutathione 2500 mg can be repeated every 8 hours for the next 24 hours or spaced out in that dose over time to decrease the side effects of any other therapeutic substances being administered and to facilitate the removal of the cell debris that will be liberated from killed cells.

The invention encompasses other variants obvious to a practitioner in the art area and equivalents to the invention. The description is not meant to be limiting in foreseeable creative variants.

### REFERENCES

1. Baay M, Brouwer A, Pauwels P, Peeters M, Lardon F. Tumor cells and tumor-associated macrophages: secreted proteins as potential targets for therapy. Clin Dev Immunol. 2011; 2011:565187. PMCID: 3227419.
2. Ohtaki Y, Ishii G, Nagai K, Ashimine S, Kuwata T, Hishida T, et al. Stromal macrophage expressing CD204 is associated with tumor aggressiveness in lung adenocarcinoma. J Thorac Oncol. 2010; 5(10):1507-15. PMID 20802348
3. Espinosa I, Jose Carnicer M, Catasus L, Canet B, D'Angelo E, Zannoni G F, et al. Myometrial invasion and lymph node metastasis in endometrioid carcinomas: tumor-associated macrophages, microvessel density, and HIF1A have a crucial role. Am J Surg Pathol. 2010; 34(11):1708-14. PMID 20962622
4. Kurahara H, Shinchi H, Mataki Y, Maemura K, Noma H, Kubo F, et al. Significance of M2-polarized tumor-associated macrophage in pancreatic cancer. J Surg Res. 2011; 167(2):e211-9. PMID 19765725
5. Fabriek B O, van Bruggen R, Deng D M, Ligtenberg A J, Nazmi K, Schornagel K, et al. The macrophage scavenger receptor CD163 functions as an innate immune sensor for bacteria. Blood. 2009; 113(4): 887-92. bloodjournal.hematologylibrary.org/content/113/4/887.long
6. Sachdev R, Robbins J, Kohler S, Vanchinathan V, Schwartz E J, Sundram U N. CD163 expression is present in cutaneous histiocytomas but not in atypical fibroxanthomas. Am J Clin Pathol. 2010; 133(6):915-21. PMID 20472850

I claim:

1. A method of treatment of idiopathic pulmonary fibrosis, comprising the following step: administering, to a patient having disease symptoms of idiopathic pulmonary fibrosis, a dose of a reduced glutathione stabilized and encapsulated in a liposomal pharmaceutical carrier capable of being ingested orally, and capable of delivering glutathione (reduced) in a physiologically active state to improve said disease symptoms by transfer of the glutathione into animal cells, where the concentration of reduced glutathione in the entrapped aqueous space of the liposomes is at least 123 mM.

2. A composition of reduced glutathione stabilized and encapsulated in a liposomal pharmaceutical carrier capable of being ingested orally, and capable of delivering glutathione (reduced) in a physiologically active state to improve said disease symptoms by transfer of the glutathione into animal cells, where the concentration of reduced glutathione in the entrapped aqueous space of the liposomes is at least 123 mM for treatment of idiopathic pulmonary fibrosis.
